(19) United States
(12) Patent Application Publication
(10) Pub. No.: US 2007/0249706 A1
(43) Pub. Date: Oct. 25, 2007

(54) USE OF CERTAIN BIPHENYL COMPOUNDS FOR PROTECTION OF NEURONS AND OLIGODENDROCYTES IN THE TREATMENT OF MULTIPLE SCLEROSIS (MS)

(75) Inventors: Jean MERRILL, Whippany, NJ (US); Sandrine FUNES, Nogent-sur-marne (FR); Wayne PETKO, South Bound Brook, NJ (US); Frederike WIRTZ-BRUGGER, Branchburg, NJ (US); Karen CHANDROSS, Somerset, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/757,607

(22) Filed: Jun. 4, 2007

Related U.S. Application Data
(63) Continuation of application No. PCT/US05/45500, filed on Dec. 14, 2005.
(60) Provisional application No. 60/640,927, filed on Dec. 31, 2004.

(51) Int. Cl. A61K 31/047; A61K 31/135; A61K 31/341; A61K 31/40

(52) U.S. Cl. 514/427; 514/461; 514/646; 514/728; 514/734

(57) ABSTRACT

A method for the treatment of multiple sclerosis through the protection on the central nervous system neurons or oligodendrocytes which comprises administering to a patient having multiple sclerosis a therapeutically effective amount of a biphenyl compound as defined by formula I as follows: 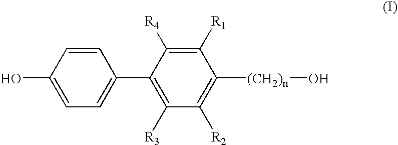
Wherein R₁, R₂, R₃ and R₄ are as defined herein

### CROSS-REFERENCE TO RELATED APPLICATIONS

This application is a continuation of International Patent Application No. PCT/US2005/045500 filed on Dec. 14, 2005 which is incorporated herein by reference in its entirety which also claims the benefit of priority of U.S. Provisional Patent Application No. 60/640,927 filed on Dec. 31, 2004.

### FIELD OF THE INVENTION

The present invention relates to methods of treating multiple sclerosis. In particular, the present invention relates to the protection of neurons and/or oligodendrocytes in multiple sclerosis patients with compounds of formula I, as well as their isomers, racemates, enantiomers, their salts, and medicaments containing them.

### BACKGROUND OF THE INVENTION

Multiple sclerosis (MS) is an autoimmune disease that leads to a loss of CNS (central nervous system) myelin, oligodendrocyte cell death and axonal destruction, causing severe functional deficits. MS occurs at a 2-3 times higher incidence in women than men (Duquette, et al., 1992.Can. J. Neurol. Sci. 19: 466-71.) and estrogen reduces disease severity during the second and third trimesters of pregnancy (Confavreux et al., 1998. N Eng J Med 339: 285-291), whereas the clinical symptoms of MS have been reported to exacerbate after delivery (Evron et al., 1984. Am. J. Reprod. Immunol. 5:109-113; Mertin and Rumjanek, 1985, J. Neurol Sci. 68:15-24; Grossman, 1989. J. Steroid Biochem. 34: 241-245; Confavreux et al., 1998, N. Engl. J. Med. 339: 285-291). Treatment with estriol decreases gadolinium enhancing lesions and MRI volume (Voskuhl and Palaszynski, 2001, Neuroscientist. 7(3): 258-270; Sicotte et al., 2002, Ann Neurol. 52: 421-428). Furthermore, estrogens cause immune response shifts, amelioration of clinical symptoms and enhanced myelin formation in rodent EAE (experimental allergic encephalomyelitis) (Curry and Heim, 1966, Nature 81: 1263-1272; Kim et al., 1999, Neurology. 52: 1230-1238; Ito et al., 2002, Clin Immunol. 102(3): 275-282). Estrogen has been reported to protect oligodendrocytes from cytotoxicity induced cell death (Takao et al., 2004. J. Neurochem. 89: 660-673); and 17β-estradiol (E2) has been reported to hasten the elaboration of multiple, interconnecting processes on oligodendrocytes (Zhang et al., 2004, J. Neurochem. 89: 674-684).

There is increasing evidence that estrogen plays a direct protective role in response to degenerative disease and injury by enhancing cell survival, axonal sprouting, regenerative responses, synaptic transmission, and neurogenesis. In the CNS, there is increased synthesis of estrogen and enhanced expression of the estrogen receptors at sites of injury (Garcia-Segura et al., 2001, Prog. in Neurobiol. 63: 29-60); and estrogen-mediated cellular protection has been demonstrated in a number of in vitro models of neurodegeneration, including β-amyloid induced cytotoxicity, excitotoxicity, and oxidative stress (Behl et al., 1995, Biochem. Biophys. Res. Commun. 216, 473-482; Goodman et al., 1996. J. Neurochem. 66:1836-1844; Green et al., 1997, J. Neurosci. 17: 511-515; BehI et al., 1999. Trends Pharmacol. Sci. 20: 441-444). Recent clinical studies suggest that estrogen replacement therapy may also decrease the risk and delay the onset and progression of Alzheimer's disease and schizophrenia. (For a review, see Garcia-Segura et al., 2001, Prog. in Neurobiol. 63: 29-60.) E2, a lipophilic hormone that can cross the blood-brain barrier, maintains the brain systems' sub-portions that are responsible for arousal, attention, mood, and cognition (Lee and McEwan, 2001. Annu. Rev. Pharmacol. & Toxicol. 41: 569-591). In addition, both natural estrogens and synthetic selective estrogen receptor modulators (SERMs), such as tamoxifen, decrease neuronal damage caused by ischemic stroke, while either E2 or raloxifene protect neurons against 1-methly-4-phenyl-1,2,3,6 tetrahydropyridine-induced toxicity (Callier, et al., 2001, Synapse 41: 131-138; Dhandapani and Brann, 2003, Endocrine 21: 59-66).

Estrogen's neuroprotective effects are mediated through the modulation of bcl-2 expression, activation of cAMP and mitogen-activated kinase signaling pathways, modulation of intracellular calcium homeostasis, enhancement of antioxidant activity, and/or activation of estrogen receptors (ER) that can act as hormone-regulated transcription factors (Mangelsdorf, et al., 1995. Cell 83: 835-839; Katzenellenbogen, et al., 1996. Mol. Endocrinol. 10: 119-131; Singer et al., 1996. Neurosci. Lett. 212: 13-16; Singer et al., 1998. Neuroreport 9: 2565-2568; Singer et al., 1999. Neurosci. Left. 212: 13-16; Weaver et al., 1997. Brain Res. 761: 338-341; Wafters and Dorsa, 1998. J. Neurosci. 18: 6672-6680; Singh et al., 1999. J. Neurosci. 19: 1179-1188; Alkayed et al., 2001. J. Neurosci. 21: 7543-7550; Garcia-Segura et al., 2001. Prog. in Neurobiol. 63: 29-60). Two characterized estrogen receptors, ERα and ERβ, belong to the class I hormone receptor family that function as nuclear transcription factors. ERα and ERβ (in the form of mRNA or protein) are expressed in neural cell types, including Schwann cells, the myelin forming cells of the peripheral nervous system, and CNS neurons, astrocytes and oligodendrocytes (Miranda and Toran-Allerand, 1992; Santagati, et al., 1994; Kuiper, et al., 1996; Mosselman, et al., 1996; Thi et al. 1998; Platania, et al., 2003). In oligodendrocytes, the myelin forming cells of the CNS that are lost in MS, ERα has been reported to be nuclear, whereas ERβ is cytoplasmic, in vivo immunoreactivity being readily detectable in cytoplasm and myelin sheaths (Zhang et al., 2004. J Neurochem 89: 674-684). Recently, Arvanitis at al., 2004 (J Neurosci Res. 75: 603-613) have reported an ER with similarities to ERβ, in isolated CNS myelin, the myelin sheath of spinal cord and brain sections and the oligodendrocyte plasma membrane.

Mimicking and/or enhancing the beneficial effects of estrogen in MS by means of small molecules that are ligands at ERβ, or compounds that preferentially mimic the effects of estrogen at sites other than the classical ERα, is likely to have advantages for the treatment of MS in that the small molecules would be devoid of the untoward “hormonal” effects of estrogen which are mediated by ERα. These other ER sites may include the recently identified ER-X, which has been identified in neurons and is developmentally regulated (Toran-Allerand 2004, Endocrinology 145:1069-1074), or GPR30, which allows estrogen to trigger different pathways that integrate cell surface signaling with gene transcription (Kanda and Watanabe 2003, J. Invest. Derm. 121: 771-780).

These compounds may also be used to treat or prevent the development of other demyelinating diseases, including Charcot-Marie-Tooth disease, Pelizaeus-Merzbacher disease, encephalomyelitis, neuromyelitis optica, adrenoleukodystrophy, Guillian-Barre syndrome, and disorders in which myelin-forming glial cells (oligodendrocytes or Schwann cells) are damaged, including spinal cord injury, neuropathies and nerve injury.

### SUMMARY OF THE INVENTION

The present invention comprises a novel use of a number of certain biphenyl compounds for the treatment of multiple sclerosis.

Broadly, the compounds of the present invention may be generically defined by the general formula (I), below: 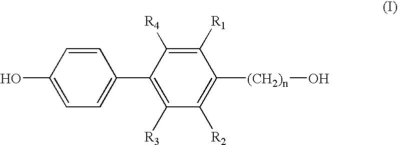
in which n is 0 or 1, R₁ represents an alkyl radical containing from 1 to 4 carbon atoms or a hydrogen atom, R₂ represents an alkyl radical containing from 1 to 4 carbon atoms or a hydrogen atom, R₃ represents a hydrogen atom; a halogen atom; an alkyl radical containing from 1 to 4 carbon atoms; an —NR_{A}R_{B} group in which R_{A} and R_{B} are identical or different and represent a hydrogen atom, or an alkyl radical containing from 1 to 4 carbon atoms; NO₂; a 5- or 6- membered cyclic or heterocyclic radical; or an alkoxy radical containing from 1 to 4 carbon atoms, R₄ represents a hydrogen atom; a halogen atom; a hydroxyl radical; an alkyl, alkenyl or alkynyl radical containing at most 4 carbon atoms; an alkoxy or alkylthio radical in which alkyl contains from 1 to 4 carbon atoms; or an —NR_{A}R_{B} group in which R_{A} and R_{B} are identical or different and represent a hydrogen atom, or an alkyl radical containing from 1 to 4 carbon atoms, its'isomers, racemates and enantiomers, and the salts of said compound derived from an inorganic or organic acid thereof.

When R₁, R₂, R₃, R₄, R_{A} and R_{B} represent an alkyl radical containing from 1 to 4 carbon atoms, it is a methyl, ethyl, propyl, isopropyl, butyl, isobutyl or tert-butyl radical. When R₃, and R₄ are a halogen atom, it is fluorine, chlorine, bromine or iodine. Preferably, it is chlorine. When R₄ is an alkenyl radical containing at most 4 carbon atoms, preferably it is a vinyl or propenyl radical. When R₄ is an alkynyl radical containing at most 4 carbon atoms, preferably it is an ethynyl or propynyl radical. When R₃ or R₄ represent an alkyloxy radical containing from 1 to 4 carbons atoms, preferably it is a methoxy, ethoxy, propyloxy, isopropyloxy or butyloxy radical. When R₄ is an alkylthio radical containing from 1 to 4 carbon atoms, preferably it is a methylthio, ethylthio, propylthio, isopropylthio or butylthio radical. When R₄ is an NR_{A} R_{B} radical in which R_{A} and R_{B} are identical or different and represent a hydrogen atom or an alkyl radical containing from 1 to 4 carbon atoms, preferably R₄ is an amino, methylamino, ethylamino, dimethylamino, diethylamino or methylethylamino radical.

The invention also comprises the acid addition salts of the compounds of formula (I), in particular when the compounds of formula (I) contain an amino function. These are the salts formed, for example, with the following acids: hydrochloric, hydrobromic, nitric, sulphuric, phosphoric, acetic, formic, propionic, benzoic, maleic, fumaric, succinic, tartaric, citric, oxalic, glyoxylic, aspartic, and alkanesulphonic acids such as methane- and ethanesulphonic acids, arenesulphonic acids, such as benzene and para-toluene sulphonic acids and arylcarboxylic acids.

These are also the salts formed under the action of a base or an alkali or alkaline-earth metal, in order to obtain, for example, derivatives such as sodium or potassium alcohol or derivatives such as potassium or sodium phenolate.

Preferably, R₁ is H; R₂ is H; R₃ is selected from alkoxy containing 4 carbon atoms, Br, Cl, pyrollyl and NZ₂, wherein Z is either CH₃ or O and R₄ is Br or Cl.

A preferred embodiment of the invention is the use of compounds of formula (I) as defined above selected from the group consisting of:

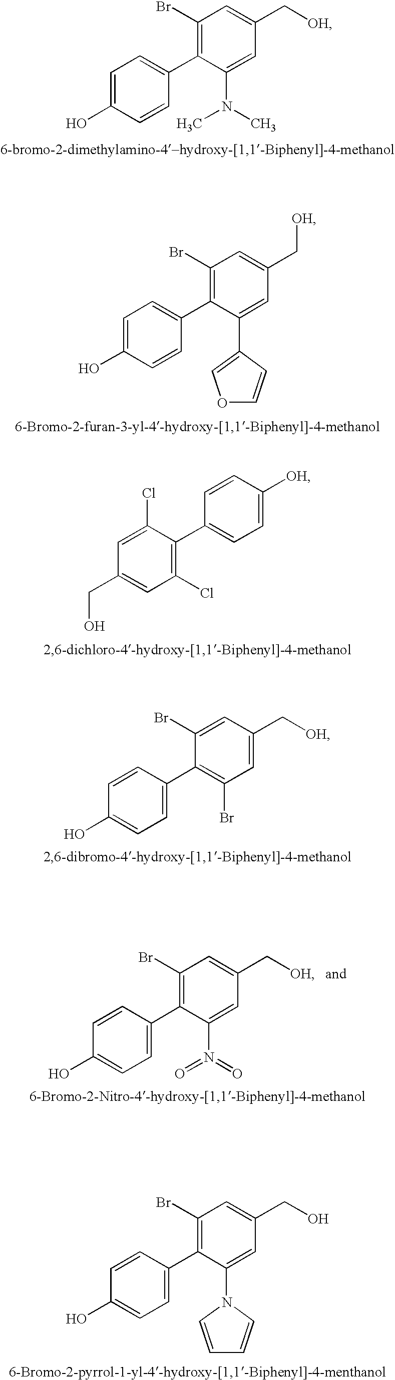

The compounds of formula (I) that contain one or more asymmetric centers have isomeric forms; these isomers and mixtures form part of the invention. The racemates and the enantiomers of these compounds also form part of the invention.

The compounds of formula I used in the process of this invention can be prepared by synthetic processes known in the art, for example, those disclosed in U.S. Pat. No. 6,147,119 to Lesuisse.

Definitions used Herein

“Pharmaceutically acceptable salts” means either an acid addition salt or a basic addition salt, whichever it is possible to make with the compounds of the present invention.

“Pharmaceutically acceptable acid addition salt” is any non-toxic organic or inorganic acid addition salt of the base compounds represented by Formula I. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents, and, in comparison to their free base forms, generally demonstrate higher melting points.

“Pharmaceutically acceptable basic addition salts” means non-toxic organic or inorganic basic addition salts of the compounds of Formula I. Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

“Patient” means a warm blooded animal, such as for example rat, mice, dogs, cats, guinea pigs, and primates such as humans. c)

“Treat” or “treating” means any treatment, including, but not limited to, alleviating symptoms, eliminating the causation of the symptoms either on a temporary or permanent basis, or preventing or slowing the appearance of symptoms and progression of the named disorder or condition. “Therapeutically effective amount” means an amount of the compound, which is effective in treating the named disorder or condition.

“Pharmaceutically acceptable carrier” is a non-toxic solvent, dispersant, excipient, adjuvant or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

“Stereoisomers” is a general term for all isomers of the individual molecules that differ only in the orientation of their atoms in space. It includes mirror image isomers (enantiomers), geometric (cis/trans) isomers, and isomers of compounds with more than one chiral center that are not mirror images of one another (diastereoisomers).

In treating a patient afflicted with a condition described above, a compound of Formula (I) can be administered in any form or mode which makes the compound bioavailable in therapeutically effective amounts, including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compositions of the present invention may be administered orally, for example, in the form of tablets, troches, capsules, elixirs, suspensions, solutions, syrups, wafers, chewing gums and the like and may contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials, which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The dosage range at which compounds of Formula I exhibit their ability to act therapeutically can vary depending upon the particular compound, the severity of the condition, the patient, the formulation, other underlying disease states that the patient is suffering from, and other medications that may be concurrently administered to the patient. Generally, the compounds of Formula I will exhibit their therapeutic activities at dosages of between about 0.001 mg/kg of patient body weight/day to about 100 mg/kg of patient body weight/day.

The contents of all publications and patents discussed herein are hereby incorporated herein by reference.

It will be appreciated that every suitable combination of the respective elements of the present invention may be interchanged with one or more of other similar, suitable components known in the art and changed in minor, non-functional respects. These additional embodiments of the invention are also regarded as falling within the scope of the claims herein. The examples detailed below are provided to better describe and more specifically set forth the elements and mechanics/operation of the present invention with reference to the drawings, but for obvious reasons cannot describe all of them. It is to be recognized that said examples therefore are for illustrative purposes only however, and should not be interpreted as limiting the spirit and scope of the invention as later recited by the claims that follow.

### EXAMPLES

### Pharmacological Study of the Products of the Invention

Binding Assay

A number of compounds were tested for ERα and ERβ binding using Panvera's Fluorescence Polarization Competition Assay Kits (cat. nos. P2698 and P2700). Briefly, ERα or ERβ were thawed on ice from −80° C. An estrogen receptor and fluorescent ligand (Fluormone™) complex was formed at a 15/1 molar ratio for ERα and at a 10/1 molar ratio for ER≈ (2X complex). Serial dilutions of the test compounds were made in assay buffer and the assay was initiated by adding 50 ul of the 2X receptor-ligand complex to 50 ul of compound solution in black 96-well plates. Zero percent competition (theoretical maximum polarization) was measured in wells containing 50 ul of buffer and 50 ul of the 2X receptor-ligand complex. The plates were incubated after gentle shaking in the dark at room temperature. Polarization values (mP) were read no longer than 7 hours after the reaction was started with a FARCyte Fluorescent reader (Amersham) at excitation and emission wavelengths of 485 nm and 535 nm, respectively. Data was analyzed using non-linear regression and IC50 values determined using GraphPad Prism. Estradiol was used as the reference compound.

Oligodendrocyte Toxicity Assay

Primary rat oligodendrocyte progenitor cells were obtained from the cerebra of 2-3 day old postnatal rats (Sprague Dawley). The meninges were removed and tissue was mechanically dissociated. Cells were plated on T75 flasks and fed with DMEM +10% FBS.

Enriched OLPs were collected by mechanical separation from the astrocytic monolayer and were expanded in serum free media (SFM) supplemented with the mitogens, PDGF-AA (10 ng/mI) and FGF-2 (10 ng/mI).

To generate mature oligodendrocytes, progenitor cells were switched to SFM supplemented with IGF-1 (10 ng/mi) 24 hours after plating and cells were grown under these conditions for 7 days prior to experimental assays.

Cells were plated in 96-well plates, 10,000 per well. Medium was changed to fresh medium and cells were pretreated with compounds for 1 hour. Toxins were added to give the following final concentrations: Sin-10 mM, Camptothecin 1 OM

Dose Response

After 24 hours, medium was removed and assayed for LDH activity using the Promega cytotox 96 kit (catalog# G1780). Results were calculated as percent protection against toxin-induced toxicity.

These compounds have been assessed for their efficacy in protection against cell death produced by toxic agents such as SIN-1 (3-morpholino-sydnonimine, producing peroxynitrite) and camptothecin. The target cells assessed in vitro are primary cultures of rodent oligodendrocyte progenitors and their mature counterparts.

Protection by these serm-like compounds have been compared to 17-β-estradiol and arzoxifen (a commercially available_SERM).

| | | ER□ selectivity | | | % Protection | |
|---|---|---|---|---|---|---|
| | | ER□ | ER□ | | (SIN-1*) | |
| | | Binding | binding | Fold | OLP | OL |
| Cpd | Structure | IC50 nM | IC50 nM | Diff | 10 nM | 10 nM |
| 2-bromo-6- (furan-3-yl)-4′- hydroxy-[1,1′- biphenyl]-4- methanol | 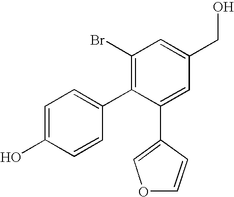 | 157 | 6 | 25 | 37 | 16 |
| 2-bromo-6- dimethylamino-4′- hydroxy-[1,1′- biphenyl]-4- methanol | 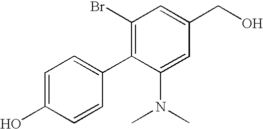 | 420 | 37 | 11 | 45 | 12 |
| 2,6-dibromo-4′- hydroxy-[1,1′- biphenyl]-4- methanol | 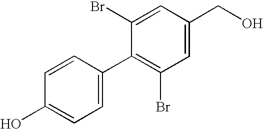 | 60 | 9 | 7 | 51 | 11 |
| 2,6-dichloro-4′- hydroxy-[1,1′- biphenyl]-4- methanol | 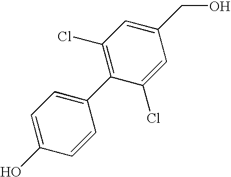 | 93 | 18 | 5 | 36 | 6 |
| 2-bromo-4′- hydroxy-6-(1H- pyrrol-1-yl)-1,1′- biphenyl]-4- methanol | 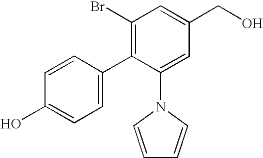 | 408 | 15 | 27 | 10 | 15 |
| 2-bromo-4′- hydroxy-6-nitro- [1,1′-biphenyl]-4- methanol | 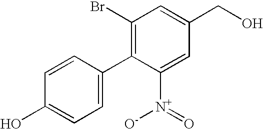 | 125 | 8 | 16 | 16 | 12 |

^{a}signifies the percentage protection obtained with 10 μM.

What is claimed is:

1. A method for the treatment of multiple sclerosis through the protection of neurons or oligodendrocytes which comprises administering to a patient having multiple sclerosis a therapeutically effective amount of a compound of Formula I, 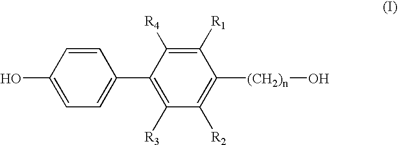
wherein n is 0 or 1, R₁ represents an alkyl radical containing from 1 to 4 carbon atoms or a hydrogen atom, R₂ represents an alkyl radical containing from 1 to 4 carbon atoms or a hydrogen atom, R₃ represents a hydrogen atom; a halogen atom; an alkyl radical containing from 1 to 4 carbon atoms; an —NR_{A}R_{B} group in which R_{A} and R_{B} are identical or different and represent a hydrogen atom, or an alkyl radical containing from 1 to 4 carbon atoms; NO₂; a 5- or 6- membered cyclic or heterocyclic radical; or an alkoxy radical containing from 1 to 4 carbon atoms, R₄ represents a hydrogen atom; a halogen atom; a hydroxyl radical; an alkyl, alkenyl or alkynyl radical containing at most 4 carbon atoms; an alkoxy or alkylthio radical in which alkyl contains from 1 to 4 carbon atoms; or an —NR_{A}R_{B} group in which R_{A} and R_{B} are identical or different and represent a hydrogen atom, or an alkyl radical containing from 1 to 4 carbon atoms, its isomers, racemates and enantiomers, and the acid addition salt of said compound.

2. The method of claim 1 wherein R₁ is H.

3. The method of claim 1 wherein R₂ is H.

4. The method of claim 1 wherein R₃ is selected from alkoxy containing 4 carbon atoms, Br, Cl, pyrollyl and NZ₂, wherein Z is either CH₂ or O.

5. The method of claim 1 wherein R₄ is Br or Cl.

6. The method of claim 1 wherein said compound of formula (I) is selected from the group consisting of:
2-bromo-6-dimethylamino-4′-hydroxy-[1,1′-Biphenyl]-4-methanol,
2-Bromo-6-furan-3-y4′-hydroxy-[1,1′-Biphenyl]-4-methanol,
2,6-dichloro-4′-hydroxy-[1,1′-Biphenyl]-4-methanol,
2,6-dibromo-4′-hydroxy-[1,1′-Biphenyl]-4-methanol,
2-Bromo-6-Nitro-4′-hydroxy-[1,1 ′-Biphenyl]-4-methanol, and
2-Bromo-6-pyrrol-1-yl -4′-hydroxy-[1,1′-Biphenyl]-4-methanol

7. The method of claim 1 wherein said effective amount is administered daily and is in the range from about 0.001 to about 100 mg/kg of patient body wt./day.

8. The method of claim 7 wherein said effective amount of the compound of Formula (I) is comprises a pharmaceutical composition which is suitable for administration to the patient orally, sublingually, buccally, subcutaneously, intramuscularly, intravenously, transdermally, intranasally, rectally, and topically
